(19) United States
(12) Patent Application Publication
(10) Pub. No.: US 2006/0084812 A1
(43) Pub. Date: Apr. 20, 2006

(54) Purification of impure materials using non-aqueous solvents

(76) Inventors: Simon Andrew Bellamy, Redhill, Surrey (GB); Lyn Hughes, Harleysville, PA (US); Mazin Nicola, Worthing, West Sussex (GB)

Correspondence Address: ROTHWELL, FIGG, ERNST & MANBECK, P.C., 1425 K STREET, N.W., SUITE 800, WASHINGTON, DC, 20005, US

(21) Appl. No.: 10/501,764

(22) PCT Filed: Jan. 18, 2002
(86) PCT No.: PCT/GB02/00201

§ 371 Date: Mar. 23, 2005

(51) Int. Cl. C07D 401/04

(52) U.S. Cl. 546/279.4

(57) ABSTRACT

An improved process for improving the purity of an impure material, for example for producing low color nicotine is disclosed. The process involves passing a solution of less pure material, for example nicotine, in a non-aqueous solvent, such as a fluorinated hydrocarbon, for example tetrafluoroethane, through an ion exchange resin or absorbent such that the impurity is retained on the resin, and purer material passes through. The process eliminates the need for distillation to prepare a purer material, for example to separate nicotine from colour causing compounds. Said process is non-flammable, non-ozone depleting, and has low toxicity.

### BACKGROUND OF THE INVENTION

This invention relates to the purification of impure materials and particularly, although not exclusively, relates to the purification of extracts of botanical materials, for example nicotine.

Nicotine is a naturally occurring alkaloid that is found in the tobacco plant, Nicotiana tobacum. It finds great use in the pharmaceutical and agricultural industries. In the pharmaceutical industry it is extensively used in smoking cessation formulations. In this use the nicotine can be administered in the form of lozenges, chewing gum, and inhalers. Because these applications are for human ingestion it is required that the nicotine be of very high purity as defined in the US Pharmacopeia. In agriculture it is used as a pesticide and is usually formulated as the nicotine sulfate salt dissolved in water. The common concentration is 40% nicotine. When used as a pesticide it is not necessary to meet the same stringent purity requirements as for pharmaceutical use.

Typically, nicotine is produced by extraction from tobacco leaves or waste products from the manufacture of tobacco for smoking. This extraction has been achieved both by extraction with organic solvents and aqueous solvents. The extraction is followed by multiple purification steps. These steps can include liquid-liquid extraction, chromatography, distillation, and ion exchange absorption/elution. For the production of high purity nicotine the final steps include vacuum distillation. The primary purpose of the distillation is to separate the nicotine from colored impurities. It also serves to reduce the water content.

The quality of the nicotine will degrade if exposed to excessive heat, or to air. In both cases the nicotine will develop a yellow to brown coloration which is not acceptable when high purity nicotine is required. Nicotine is not a volatile compound (bp 247° C.@ 745 mm Hg) and very low pressures are typically used to prevent the use of excessive heat. The exposure to air can lead to shortened shelf life of high purity nicotine.

Ion exchange resins have been used for the purification of nicotine at different stages in its isolation. Said purification work was done in aqueous solutions. However, the use of aqueous solutions can lead to the need for removal of large quantities of water to make solutions of concentrations suitable for use in the industry, e.g., as a 40% solution of nicotine sulfate as an agricultural pesticide. The removal of the water can be a large energy cost and can produce hazardous waste. When ion exchange resins have been used in the purification of nicotine it is required that the nicotine be absorbed onto the resin. The nicotine is first extracted with the ion exchange resin and absorbed onto the resin and then it is eluted from said resin. See, Prabhu et al, Tobacco Research 18, 125-128, 1992; Narasimha et al, Research and Industry 37, 115-117, 1992; French patent 1473458; Badgett, Ind Eng Chem, 42(12) 1950, 2530-1; Bhat et al, Proc Nat Acad Sci India, 60(a) IV, 359-362, 1990; de Lucas, Ind Eng Chem Res, 37, 4783-4791, 1998.

Attempts have been made to decolorize with non-aqueous solvents. Specifically, Walden and Gregor (Principles and Applications of Water Chemistry, Proceedings of the Rudolfs Research Conference, 4^{th} 491-504, 1965), reported the results of using ion exchange resins with nicotine in non-aqueous systems. Their resins were strongly acidic cation exchange resins with a styrenic backbone and with pendant lauryl chains to increase lipophilicity. Use of an exotic eluant, 0.02N n-butylamine in n-heptane, enabled them to separate aniline and nicotine. Because of the use of n-butylamine, this approach is impractical for industrial scale purification. Also, CN1136563A, describes a multistep purification process whereby the aqueous extract is first treated with sulfuric acid to precipitate inorganic salts, then it is passed over a porous, strongly acidic cation exchange resin with a styrenic backbone, to absorb the nicotine. Elution is achieved with ammonium hydroxide mixed with sodium or potassium hydroxide. After concentrating the eluate by distillation it is then extracted with benzene. The final benzene solution is 40%. Removal of the benzene by distillation gives pure, colorless nicotine.

In a preferred embodiment of the present invention relating to nicotine purification, the impurities, e.g., the undesirable color-causing components, are preferentially held by the ion exchange resin or adsorbent, and low color nicotine is produced. The process used may represent an improvement in the art because it does not involve distillation or the use of high temperature. It also has the potential for point-of-use application to avoid storage problems. Further, Applicants' most preferred non-aqueous solvent is non-toxic to humans. It is approved by the FDA for use as the propellant in inhalers. Many of the solvents are non-ozone depleting and non-flammable.

The following terms have the following meanings herein:

The term “water retention capacity,” as used herein, is used to describe the maximum amount of water that an ion exchange resin can retain within the polymer phase and in any pores. (ASTM D2187: Standard Test Methods for Physical and Chemical Properties of Particulate Ion Exchange Resin. Test Method B: Water Retention Capacity)

The term “acrylic” as used herein is used to describe polymers of acrylic acid and its esters, and methacrylic acid and its esters, regardless of their method of manufacture.

The term “aromatic” as used herein is used to describe polymers of aromatic monomers, such as divinyl benzene, styrene, and ethylvinyl benzene.

Further, ion exchange resins are characterized by their capacity to exchange ions. This is expressed as the “Ion Exchange Capacity.” For cation exchange resins the term used is “Cation Exchange Capacity.” The ion exchange capacity is measured as the number equivalents of an ion that can be exchanged and can be expressed with reference to the mass of the polymer (herein abbreviated to “Weight Capacity”) or its volume (often abbreviated to “Volume Capacity”). A frequently used unit for weight capacity is “milliequivalents of exchange capacity per gram of dry polymer.” This is commonly abbreviated to “meq/g.”

The term “adsorbents,” as used herein, means porous materials that are characterized by their surface area, pore size, and surface functionality. The surface area is typically expressed in terms of area per dry weight, such as “m²/g”. Pore size is expressed in terms of the pore diameter, for example ‘nanometers’ or ‘Angstroms’. Surface properties are related to chemical composition.

### STATEMENT OF THE INVENTION

According to a first aspect of the invention, there is provided a method of improving the purity of an impure material comprising the steps of:
a) selecting a solution which comprises said impure material and a non-aqueous solvent;
b) contacting said solution with an ion exchange resin or adsorbent so that said resin or adsorbent removes impurities from the impure material;
c) collecting solution after contact with said resin or adsorbent in step b); and
d) removing said non-aqueous solvent from the solution collected in step c), thereby leaving a material of improved purity.

### DETAILED DESCRIPTION OF THE INVENTION

Ion exchange resins useful in the practice of the present invention include, but are not limited to, anionic exchange resins and cationic exchange resins.

Preferred anionic exchange resins include, but are not limited to, styrenic strongly basic anion exchange resins with a quaternary amine functionality having a weight capacity of 0.1 to 15 meq/g, and styrenic weakly basic anion exchange resins with a primary, secondary, or tertiary amine functionality having a weight capacity of 0.1 to 8.5 meq/g, and acrylic or methacrylic strongly basic anion exchange resins with a quaternary amine functionality having a weight capacity of 0.1 to 12 meq/g, and acrylic or methacrylic weakly basic anion exchange resins with a primary, secondary, or tertiary amine functionality having a weight capacity of 0.1 to 12 meq/g, and allylic and vinylic weakly basic anion exchange resins with a primary, secondary, or tertiary amine functionality having a weight capacity of 0.1 to 24 meq/g.

Most preferred anionic exchange resins include, but are not limited to, styrenic anion exchange resins with quaternary amine functionality with weight capacity of 0.1 to 6 meq/g and acrylic anion exchange resins with tertiary amine functionality with weight capacity of 0.1 to 12 meq/g.

Cationic exchange resins useful in the practice of the present invention include, but are not limited to, styrenic strongly acidic cation exchange resins with sulfonic or phosphonic acid functionalities having a weight capacity of 0.1 to 8 meq/g, styrenic weakly acidic cation exchange resins with carboxylic or phenolic acid functionalities having a weight capacity of 0.1 to 8.5 meq/g, or acrylic or methacrylic weakly acidic cation exchange resins with a carboxylic acid functionality with weight capacity of 0.1 to 14 meq/g.

Preferred cationic exchange resins include, but are not limited to, styrenic weakly acidic cation exchange resin with a phenolic acid functionality with a weight capacity of 0.1 to 8.5 meq/g or, a styrenic strongly acidic cation exchange resin with a sulfonic acid functionality with weight capacity of 0.1 to 8 meq/g, and acrylic or methacrylic weakly acidic cation exchange resin with a carboxylic acid functionality with weight capacity of 0.1 to 14 meq/g.

The more preferred cationic exchange resins include, but are not limited to, acrylic or methacrylic weakly acidic cation exchange resin with a carboxylic acid functionality with weight capacity of 0.1 to 14 meq/g.

The most preferred cationic exchange resins are methacrylic weakly acidic cation exchange resin with a carboxylic acid functionality with weight capacity of 0.1 to 12 meq/g.

Strongly acidic and weakly acidic cation exchange resins useful in the practice of the present invention are in the acid form or salt form or partial salt form.

Weakly basic anion exchange resins useful in the practice of the present invention are in the free base form or salt form or partial salt form.

Ion exchange resins are manufactured in different forms. These forms can include spherical and non-spherical particles with size in the range of 0.001 mm to 2 mm. The non-spherical particles are frequently manufactured by grinding of the spherical particles. Products made in this way typically have particle size in the range 0.001 mm to 0.2 mm. The spherical particles are frequently known in the art as “Whole Bead.” The non-spherical particles are frequently known in the art as “Powders.”

Adsorbents useful in the practice of this invention include, but are not limited to, carbonaceous adsorbents, acrylic adsorbents, phenol-formaldehyde adsorbents, silica, and alumina.

Preferred adsorbents useful in the practice of this invention are carbonaceous adsorbents, acrylic adsorbents, and phenol-formaldehyde adsorbents.

More preferred adsorbents useful in the practice of this invention are acrylic adsorbents adsorbents.

Ion exchange resins and adsorbents useful in this invention are in powder or whole bead form.

The preferred ion exchange resins and adsorbents useful in this invention are in powder form or small particle size whole bead form.

Preferred ion-exchange resins and adsorbents useful in this invention are polymeric resins. Thus, preferred resins and adsorbents are organic polymers.

Ion-exchange resins may be a preferred material for use in the method.

The ion exchange resins useful in this invention have an amount of water between 0% and the water retention capacity of said resin.

The preferred ion exchange resins used in the invention have between 0% and 25% water.

The most preferred ion exchange resins used in the invention have between 0% and 10% water.

The solvents that can be used in the invention are non-aqueous solvents including, but not limited to, halogenated hydrocarbons, ketones, alcohols, ethers, hydrocarbons, esters, nitrites, and mixtures thereof. The preferred non-aqueous solvents useful in the present invention are fluorinated hydrocarbon solvents.

A preferred fluorinated hydrocarbon is a C₁ to C₄ fluorinated hydrocarbon. The C₁ to C₄ fluorinated hydrocarbon may be non-chlorinated. Preferably, it comprises one or more carbon, fluorine and hydrogen atoms only. Preferably, said fluorinated hydrocarbon is a C₁ to C₃, more preferably, a C₁ to C₂ fluorinated hydrocarbon. Especially preferred is a C₂ fluorinated hydrocarbon.

Said fluorinated hydrocarbon may include up to 10, preferably up to 8, more preferably up to 6, especially up to 4, fluorine atoms.

Said fluorinated hydrocarbon is preferably aliphatic. It is preferably saturated.

Said fluorinated hydrocarbon may have a boiling point at atmospheric pressure of less than 20° C., preferably less than 10° C., more preferably less than 0° C., especially less than −10° C. The boiling point may be greater than −90° C., preferably greater than −70° C., more preferably greater than −50° C.

Preferred non-aqueous solvents comprise:
trifluoromethane (CF₃H);
fluoromethane (CH₃F);
difluoromethane (CF₂H₂);
1,1-difluoroethane (CF₂HCH₃);
1,1,1-trifluoroethane (CF₃CH₃);
1,1,1,2-tetrafluroethane (CF₃CFH₂) (TFE)
pentafluoroethane (CF₃CF₂H);
1,1,1,2,2-pentafluorpropane (CF₃CF₂CH₃);
1,1,1,2,2,3-hexafluoropropane (CF₃CF₂CFH₂);
1,1,1,2,3,3-hexafluoropropane (CF₃CFHCF₂H);
1,1,1,3,3,3-hexafluropropane (CF₃CH₂CF₃);
1,1,2,2,3,3-hexafluoropropane (CF₂HCF₂CF₂H);
1,1,1,2,2,3,3-heptafluoropropane (CF₃CF₂CF₂); 1,1,1,2,3,3,3-heptafluoropropane (CF₃CFHCF₃). Tetrafluoroethane is an especially preferred non-aqueous solvent with 1,1,1,2-tetrafluoroethane (TFE) (CF₃CFH₂), being most preferred.

Said non-aqueous solvent of said solution may include a fluorinated hydrocarbon solvent (especially TFE) as described together with one or more co-solvents. Said solvent may include less than 20 wt %, preferably less than 15 wt %, more preferably less than 10 wt % of co-solvent.

A said co-solvent may be selected from: a C₂₋₆ hydrocarbon such as an alkane or cycloalkane with alkanes such as ethane, n-propane, i-propane, n-butane and i-butane being especially preferred; and hydrocarbon ethers, particularly dialkylethers such as dimethylether, methylethylether and diethyl ether. In other embodiments, said co-solvent may be polar, for example having a dielectric constant, at 20° C., of greater than 5. Such co-solvents may be selected from:

amides, especially N,N′-dialkylamides and alkylamides, with dimethylformamide and formamide being preferred; sulphozides, especially dialkyl sulphoxides, with dimethylsulphoxide being preferred; alcohols, especially aliphatic alcohols for example alkanols, with methanol, ethanol, 1-propanol and 2-propanol being preferred; ketones, especially aliphatic ketones, for example dialkyl ketones, with acetone being especially preferred; organic acids, especially carboxylic acids with formic acid and acetic acid being preferred; carboxylic acid derivatives, for example anhydrides, with acetic anhydride being preferred; cyanide derivatives, for example hydrogen cyanide and alkyl cyanides, with methyl cyanide and liquefied anhydrous hydrogen cyanide being preferred; ammonia; sulphur containing molecules including sulphur dioxide, hydrogen sulphide and carbon disulphide; inorganic acids for example hydrogen halides with liquefied anhydrous hydrogen fluoride, chloride, bromide and iodide being preferred; nitro derivatives, for example nitroalkanes and nitroaryl compounds, with nitromethane and nitrobenzene being especially preferred.

In a preferred embodiment when a fluorinated hydrocarbon solvent is used, substantially no co-solvent of the types described is used. Preferably, therefore, said solution selected in step (a) of the method consists essentially of a said fluorinated hydrocarbon solvent (especially TFE).

Preferably, said solution contacted with said ion-exchange resin or adsorbent in step (b) does not include any chlorinated hydrocarbon solvent.

The method of the first aspect may include the step of preparing a said solution comprising said impure material and a said non-aqueous solvent for use in step (b). Thus, the method may include contacting a said solution and a said impure material prior to step (b). Thus, suitably, the solution is prepared and, subsequently, is contacted with ion-exchange resin or adsorbent in step (b).

An impure material for treatment in the method as described above may be a naturally-occurring material and/or a material derived from a natural source or a synthetic material. A said naturally-occurring material may be an extract from a botanical material. Preferred extracts include nutraceutical and biologically active extracts of botanical materials and flavours and fragrances.

Nutraceutical and Biologically active extracts of botanical materials that may be purified include, but are not limited to, anti-oxidants such as phytophenols obtained from extracts of rosemarinus officinalis, anti-fungal and anti-infective agents such as extracts of oregano and Cocos mucifera, carminatives such as extracts of peppermint, malaria therapeutics such as artemisinins from extracts of Artemesia annua, anxiolytic agents such as Kavalactones from Piper methysticum (e.g. kava root), ACE and AchE enzyme inhibition agents such as extracts of ginseng and salvia, cytotoxic agents such as extracts of cowbane and Bullatacinone obtained from extract of Annona bullata, analgesics such as extracts of Mentha piperita, anticeptics, such as extracts of Mentha piperita, pheromone such as heptan-2-one from extract of Arum maculatum, sedatives such as linalool from extract of Coriandum sativum, vascodilators such as theobromine from the extract of Theobroma cacao and histamine from musa sapientum (banana plant), anaesthetic such as Aconitine from the extract of Aconitium napellus.

Flavours and fragrances that are useful include, but not limited to, menthol from the extract of Mentha piperita, gamma-nonalactone from Prunus persica, Linalool from extracts of Coriandum sativum, geranyl acetate from extract of Pelagonium odoratissimum, Jasmone from extracts of Jasminum officinalis, extract of rose flower, cinnamaldehyde from extracts of cinnamon, extracts of vanilla, whole extract of peppermint and spearmint.

Favors and fragrances which may be useful and can be obtained synthetically include, but are not limited to, vanillin, methyl salicylate, thymol and ethyl vanillin.

An impure material for treatment in the method may be a derivative of a botanical extract.

Extracts for treatment in the method may be prepared as described in EP 94301199.9 and WO95/26794, the contents of which are incorporated herein by reference.

The material of improved purity produced in step (d) is preferably a purified form of an extract from a botanical material. Said material of improved purity may be a nutraceutrical and/or biologically active extract of a botanical material and/or a flavour or fragrance. Said material of improved purity preferably does not comprise an organic material which is made up of carbon and hydrogen atoms only.

Preferably, impurities removed in step (b) are not enantiomers of any material of improved purity produced in step (d).

A suitable concentration of the impure material to non-aqueous solvent in said solution described in step (a) is from 0.01% to 40% by weight of impure material.

A preferred concentration of impure material to non-aqueous solvent is from 0.1% to 20% by weight of impure material.

A more preferred concentration of impure material to non-aqueous solvent is from 0.5% to 10% by weight of impure material.

The most preferred concentration of impure material to non-aqueous solvent in said solution described in step (a) is from 1% to 10% by weight.

The range of ratios of impure material to ion exchange resin or adsorbent contacted in step (b) is suitably 0.05:1 to 500:1 by weight.

The preferred range of ratios of impure material to ion exchange resin is 0.2:1 to 250:1 by weight.

The more preferred range of ratios of impure material to ion exchange resin or adsorbent contact in step (b) is 0.5:1 to 50:1 by weight.

The mode of operation of the present invention can be batch operation or column operation. Where the method involves batch operation, ion-exchange resin or adsorbent may be contacted with solution in a container which has a closed end so that solution can be held in the container and cannot pass through from one side to an opposite side. The amount of solution contacted with resin or adsorbent in step (b) may be at least 5 ml per gram of resin/adsorbent, preferably at least 10 ml per gram of resin/adsorbent, especially at least 13 ml per gram of resin/adsorbent.

Where the method involves column operation, the resin/adsorbent is suitably packed in a column between respective opposite open ends thereof. The rate of flow of solution through the column in step (b) may be at least 5 ml per hour per gram of resin/adsorbent, preferably at least 10 ml per hour per gram of resin/adsorbent and, more preferably, at least 15 ml per hour per gram of resin/adsorbent. The rate may be less than 100 ml per hour per gram of resin/adsorbent.

The method may be operated on an industrial scale, suitably using at least 20 g, preferably at least 50 g, more preferably at least 0.5 kg, especially at least 1 kg of resin/adsorbent in step (b). Suitably at least 1 litre, preferably at least 5 litres, especially at least 10 litres of solution is contacted with resin/adsorbent in step (b).

In a preferred embodiment of the invention, there is provided a method for purifying nicotine comprising the steps of:
a. dissolving nicotine in a non-aqueous solvent to form a nicotine/non-aqueous solvent solution;
b. passing said solution formed in step a. through an ion exchange resin or adsorbent to obtain a solution with reduced color,
c. evaporating said non-aqueous solvent from said solution obtained in step b. to obtain a low color nicotine.

Specifically when using a non-aqueous solvent, such as 1,1,1,2-tetrafluoroethane (TFE), the colored, impure nicotine, is charged to a suitable vessel, and then said vessel is evacuated to remove the air. TFE is then added, and the pressure is allowed to rise to the vapor pressure of the TFE (approximately 520 kPascals at room temperature) to maintain the TFE in the liquid state. The nicotine dissolves in the TFE, and then, while still under pressure, the nicotine and TFE are passed over a suitable ion exchange resin or adsorbent. The color is retained on the said resin or adsorbent and the effluent nicotine solution is essentially colorless. TFE is then removed from the solution by reducing the pressure slowly, and providing a heat source to maintain the temperature of the solution between room temperature and the boiling point of TFE. A temperature near room temperature is preferred to remove the TFE quickly. Because TFE has such a low boiling point it is removed essentially quantitatively at atmospheric pressure. The TFE can be recovered and reused by using a compressor and condenser, or a condenser at less than the boiling point of the TFE. The resulting nicotine has low color and contains less impurities than the starting nicotine.

The ion exchange resin or adsorbent used in this invention can be regenerated for re-use using any of the regeneration methods known in the art, such as treatment with a strong acid or washing with solvents.

Because of the simplicity of the invention it can be used as a point-of-use method for purifying impure materials such as colored nicotine, either because said nicotine was originally colored, or because it has developed color during storage.

The present invention is also useful when combined with a solvent-based, for example TFE based, process for preparing resinates as described in an application, entitled: A Method for Preparing Resinates, filed concurrently with the present application. In one embodiment, a TFE/nicotine solution after purification as described herein can be used directly for the loading process without need to evaporate the TFE. This combination has the advantage that the colored nicotine can be used as the raw material for the process. The colored nicotine is significantly cheaper than high purity nicotine.

The present invention can also be used in combination with the extraction of nicotine from an aqueous extract of tobacco or tobacco products using TFE as taught in WO98/45013, or those solvents currently used in the art. The method of aqueous extraction is well known in the art. In this combination the TFE or said other solvent extract is passed immediately through the ion exchange resin without evaporating the TFE or said other solvent. This methodology may be extended to other extracts, for example other botanical extracts. Nicotine useful in the practice of the present invention includes, but is not limited to that derived from the extraction of nicotine from the tobacco plant Nicotiana tobacum, and nicotine from any source that has developed color during storage.

The ion exchange resin or adsorbent useful in the practice of this invention can be new or regenerated.

The invention extends to a purified product of a process described herein. The following non-limiting examples illustrate the practice of the present invention.

Description of the resins used in the examples are provided in Table 1.

TABLE 1

| Resin | Description | Wt Cap | SA |
|---|---|---|---|
| I | Acrylic polymeric adsorbent | | 480 |
| II | Acrylic polymeric adsorbent, small particle size | | 480 |
| III | Aromatic polymeric adsorbent | | 550 |
| IV | Carbonaceous adsorbent | | 550 |
| V | Phenolic polymeric adsorbent | | 200 |
| VI | Strongly acidic styrenic cation exchange resin | 5.2 | |
| VII | Strongly acidic styrenic cation exchange resin | 4.1 | |
| | powder | | |
| VIII | Strongly basic styrenic anion exchange resin | 4.2 | |
| IX | Strongly basic styrenic anion exchange resin | 4.3 | |
| | powder | | |
| X | Weakly acidic acrylic cation exchange resin | 11.0 | |
| XI | Weakly acidic methacrylic cation exchange resin | 10.6 | |
| XII | Weakly acidic methacrylic cation exchange resin | 8.6 | |
| | powder | | |
| XIII | Weakly acidic methacrylic cation exchange resin | 10.6 | |
| | powder | | |
| XIV | Weakly basic acrylic anion exchange resin | 6.0 | |

### EXAMPLE 1

### Anhydrous Batch Decolorization

A series of different dry ion exchange resins and adsorbent resins were used to decolorize nicotine using the following procedure. A 1.35% solution of colored nicotine in TFE was added to the test resin in a pressure vessel equipped with a filter. The amount of solution used was 15 ml per gram of resin. The mixture was shaken for 6 hours and then filtered. The TFE was then removed from the filtered solution by slowly reducing the pressure. The resulting nicotine was evaluated for color by one person using a scale of 0-10 where 0 represented water-white and 10 represented the original color of the nicotine. The results of this experiment are shown in Table 2.

### EXAMPLE 2

### Hydrated Batch Decolorization

The procedure of Example 1 was repeated except that the test resins were fully hydrated before adding the nicotine solution.

The results of this experiment are shown in Table 2.

TABLE 2

| Resin | Example 1 | Example 2 |
|---|---|---|
| I | 1 | 3 |
| III | 2 | 2 |
| IV | 1.5 | 1 |
| V | 2 | 9 |
| VI | 5 | |
| VII | | 2 |
| VIII | 8 | 3 |
| IX | 6 | 2 |
| X | 5 | |
| XI | 6 | 9 |
| XII | 6 | 7 |
| XIII | 6 | 9 |
| XIV | 7 | 7 |

### EXAMPLE 3

### Decolorization in Column Mode

A column suitable for use under pressure was packed with 20 ml (8.8 g) of Resin II. 740 ml of 3.1% nicotine solution in TFE was passed through the column at a rate of approximately 140 ml/hour. The TFE was evaporated by reducing the pressure and then recovered by recompression. The product nicotine was very pale yellow in color. The total nicotine treated was 18.2 g.

The resin was then regenerated in place by passing a 10% solution of methanol in TFE through the column until the effluent was colorless. The regeneration solvent was recovered by evaporation/recompression. The column was then put back in service and used to treat a further 1000 ml of 3.1% nicotine solution in TFE. The nicotine so produced was very pale yellow in color. The total nicotine treated was 31.7 g.

The resin was regenerated again as above, and then used to treat a further 1200 ml of 3.1% nicotine solution. The nicotine so produced was very pale yellow in color. The total nicotine treated was 36.8 g.

The total amount of nicotine treated in these three runs was 86.7 g.

This example clearly demonstrates the ability of the invention to be used in column mode, and that it can be very effectively regenerated and re-used without loss of performance. The only waste generated were the impurities that were removed. There was no solvent waste.

1. A method of improving the purity of an impure material comprising the steps of:
a) selecting a solution which comprises said impure material and a non aqueous solvent;
b) contacting said solution with an ion exchange resin or adsorbent so that said resin or adsorbent removes impurities from the impure material;
c) collecting solution after contact with said resin or adsorbent in step b); and
d) removing said non-aqueous solvent from the solution collected in step c), thereby leaving a material of improved purity.

2. A method according to claim 1, wherein said non-aqueous solvent is a halogenated hydrocarbon, ketone, alcohol, ether, hydrocarbon, ester, nitrile or a mixture thereof.

3. A method according to claim 1, wherein said non-aqueous solvent is a fluorinated hydrocarbon solvent.

4. A method according to claim 1, wherein said non-aqueous solvent comprises tetrafluroethane.

5. A method according to claim 1, wherein said non-aqueous solvent includes a fluorinated hydrocarbon solvent together with one or more co-solvents.

6. A method according to claim 1, which includes preparing a said solution comprising said impure material and a said non-aqueous solvent which comprises a fluorinated hydrocarbon solvent prior to step (b) and, subsequently, contacting the solution prepared with ion-exchange resin or adsorbent in step (b).

7. A method according to claim 1, wherein said impure material comprises a naturally-occurring material, a material derived from a natural source or a synthetic material.

8. A method according to claim 1, wherein said impure material comprises an extract from a botanical material.

9. A method according to claim 1, wherein said impure material comprises a nutraceutical or biologically active extract of a botanical material, a flavour or a fragrance.

10. A method according to claim 1, wherein the concentration of impure material to non-aqueous solvent in said solution described in step (a) is from 0.01% to 40% by weight of impure material.

11. A method according to claim 1, wherein the range of ratios of impure material to ion-exchange resin or adsorbent contacted in step (b) is 0.05:1 to 500:1 by weight.

12. A method for purifying nicotine comprising the steps of
(a) dissolving nicotine in a non-aqueous solvent to form a nicotine/non-aqueous solvent solution;
(b) passing said solution formed in step (a) through an ion exchange resin or adsorbent to obtain a solution with reduced colour;
(c) evaporating said non-aqueous solvent from said solution obtained in step (b) to obtain a low colour nicotine.

13. A method according to claim 12, wherein said non-aqueous solvent is 1,1,1,2-tetrafluoroethane or a mixture of 1,1,1,2-tetrafluoroethane with a non-aqueous solvent selected from the group consisting of ketones, alcohols, ethers, hydrocarbons, esters or nitrites or mixtures thereof.
